(19) United States
(12) Patent Application Publication
(10) Pub. No.: US 2023/0119859 A1
(43) Pub. Date: Apr. 20, 2023

(54) APPARATUS AND A METHOD FOR DETERMINING CHARACTERISTICS OF A FLUID

(71) Applicant: EZERX HEALTH TECH PRIVATE LIMITED, Kolkata (IN)

(72) Inventors: Partha Pratim Das MAHAPATRA, Kolkata (IN); Sandeep SHARMA, New Delhi (IN)

(73) Assignee: EZERX HEALTH TECH PRIVATE LIMITED, Kolkata (IN)

(21) Appl. No.: 17/916,995

(22) PCT Filed: Apr. 5, 2021
(86) PCT No.: PCT/IN2021/050336

§ 371 Date: Oct. 4, 2022

(30) Foreign Application Priority Data
Apr. 4, 2020 (IN) 202021015008

(51) Int. Cl. A61B 5/145; A61B 5/1455

(52) U.S. Cl. CPC A61B 5/14532; A61B 5/14552; A61B 5/6826

(57) ABSTRACT

The present disclosure discloses an apparatus for determining characteristics of a fluid. The apparatus includes a visible light producing LED array, an optical system, and a microcontroller. In the apparatus, the visible light producing LED array emits light and produces a light beam for irradiating an object. Further, in the apparatus, the optimal system includes a grating to receive irradiated light from the object through a collimator and disperse the light into wavelengths, a focusing lens and a linear image sensor arranged at a focal plane of the focusing lens to convert the light by the grating and focused by the focusing lens, into electrical signals. Lastly, in the apparatus, the microcontroller is connected to the sensor and processes the electrical signals and communicates for processing.

### FIELD OF INVENTION

The present invention relates to a non-invasive portable device, more specifically, a non-invasive portable system and method for measuring user's characteristics of a fluid, such as hemoglobin, bilirubin and oxygen saturation from the human body.

### BACKGROUND OF THE INVENTION

One of the widely used methods to obtain blood samples for measuring the blood. characteristics is the invasive method. It involves piercing the skin, typically the finger to draw a drop of blood and then manually transfer it onto a disposable chemical strip. Thereafter, the blood sample is tested. During this process, the pathologist collects the blood. sample and go for the pathological test to measure blood characteristics which is very expensive and time consuming process. Further, the manual blood drawing and transferring may contaminate the sample and possibly produces incorrect results. In addition, these “invasive” methods are inconvenient and potentially even painftil for patients.

Therefore, in order to overcome the aforesaid problems, the present invention provides a non-invasive portable apparatus and method for determining characteristics of a fluid.

### SUMMARY OF THE INVENTION

This summary is provided to introduce a selection of concepts, in a simplified format, that are further described in the detailed description of the invention. This summary is neither intended to identify key or essential inventive concepts of the invention and nor is it intended for determining the scope of the invention.

In an embodiment, the present invention discloses an apparatus for determining characteristics of a fluid. The apparatus includes a visible light producing LED array, an optical system, and a microcontroller. In the apparatus, the visible light producing LED array emits light and produces a light beam for irradiating an object. Further, in the apparatus, the optimal system includes a grating to receive irradiated light from the object through a collimator and disperse the light into wavelengths, a focusing lens and a linear image sensor arranged at a focal plane of the focusing lens to convert the light by the grating and focused by the focusing lens, into electrical signals. Lastly, in the apparatus, the microcontroller is connected to the sensor and processes the electrical signals and communicates for processing.

In an embodiment, the present disclosure discloses a method of determining characteristics of a fluid. The method includes emitting light by a visible light producing LED array and producing a light beam for irradiating an object. The method includes receiving, from a grating of an optimal system, an irradiated light from the object through a collimator and dispersing the light into wavelengths. The method includes converting the optical signals into electrical signals, by a linear image sensor arranged at a focal plane of a focusing lens in the optimal system by the grating and focussing by the focusing lens. The method includes processing the electrical signals by a microcontroller connected to the sensor and communicating for processing.

In an embodiment of the present disclosure, a non-invasive portable system mainly comprises a device, a platform on mobile device having a data model, and a database. The device further comprises a light ring with an LED array to emit light and a photo diode, a microcontroller and an Analog to Digital (ADC) converter. In the system, the light ring in the device passes a light to the inner side of the ring finger of a user. The light from the light ring then touches the ring finger and is reflected at 90 degrees back to the photo diode of the light ring. The photo diode then produces a spectrum, for example, the photo diode captures more than hundreds of signals continuously with a minimum delay of 500 milliseconds. Thereafter, an average of the signals is determined by the microcontroller of the device and then it is amplified through the ADC to obtain a spectrum of signal. This signal is then sent to the platform by the device on the mobile device for further processing, say via Bluetooth.

In the platform, the signal is then compared with the reference spectrum of the database and the compared result is fed into the data model. The data model having probability factors using existing calibrated data generates the value of the haemoglobin, bilirubin and oxygen saturation of the user's blood from the signal. Lastly, these values of user's health parameters are stored in the cloud for future references.

In an embodiment of the present disclosure, a method of working of a non-invasive portable system includes, passing a light, by a light ring in a device, to the inner side of the ring finger of a user. The method includes reflecting the light at 90 degrees back from the ring finger to the photo diode of the light ring. The method includes producing a spectrum by the photo diode, for example, the photo diode captures more than hundreds of signals continuously with a minimum delay of 500 milliseconds. The method includes determining an average of the signals by the microcontroller of the device and then amplifying the signals through the ADC to obtain a spectrum of signal. The method includes sending the signal to the platform by the device on the mobile device for further processing, say via Bluetooth.

The method includes comparing the signal with the reference spectrum of the database in the platform and then feeding the compared result into the data model. The method includes generating the value of the haemoglobin, bilirubin and oxygen saturation of the user's blood from the signal using the data model having probability factors using existing calibrated data. Lastly, the method includes storing these values of user's health parameters in the cloud for future references.

The advantages of the present invention are:

1. Non-invasive and non-contact for painless and infection free process.

2. Affordable and accessible.

3. IoT enabled system.

4. Portable and battery operated.

5. Easy to operate.

To further clarify advantages and features of the present invention, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 illustrates an apparatus for determining characteristics of a fluid, in accordance with the present disclosure;

FIG. 2 illustrates working of an optical system in the apparatus, in accordance with the present disclosure;

FIG. 3 illustrates a digital diagram of the electrical signals obtained as an output of the apparatus, in accordance with the present disclosure;

FIG. 4 illustrates a method of determining characteristics of a fluid, in accordance with the present disclosure;

FIG. 5 illustrates an example embodiment of the apparatus, in accordance with the present disclosure;

FIG. 6 illustrates the data processing steps for generation to output, in accordance with the present disclosure; and

FIG. 7 illustrates auto-generated rules used in the data model, in accordance with the present disclosure.

Further, skilled artisans will appreciate that elements in the drawings are illustrated for simplicity and may not have been necessarily drawn to scale. For example, the flow charts illustrate the method in terms of the most prominent steps involved to help to improve understanding of aspects of the present invention. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having benefit of the description herein.

### DETAILED DESCRIPTION OF FIGURES

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are explanatory of the present disclosure and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skilled in the art to which this disclosure belongs. The system, methods, and examples provided herein are illustrative only and not intended to be limiting.

Embodiments of the present invention will be described below in detail with reference to the accompanying drawing.

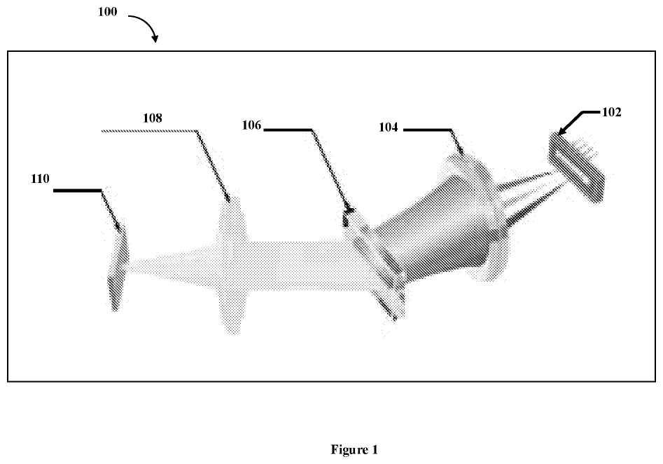

The present disclosure relates to a non-invasive portable system mainly comprises a device, a platform on mobile device having a data model, and a database, as shown in FIG. 1. The device further comprises a light ring with an LED array to emit light and a photo diode, a microcontroller and an Analog to Digital (ADC) converter. In the system, the light ring in the device passes a light to the inner side of the ring finger of a user. The light from the light ring then touches the ring finger and is reflected at 90 degrees back to the photo diode of the light ring. The photo diode then produces a spectrum, for example, the photo diode captures more than hundreds of signals continuously with a minimum delay of 500 milliseconds. Thereafter, an average of the signals is determined by the microcontroller of the device and then it is amplified through the ADC to obtain a spectrum of signal. This signal is then sent to the platform by the device on the mobile device for further processing, say via Bluetooth.

In the platform, the signal is then compared with the reference spectrum of the database and the compared result is fed into the data model. The data model having probability factors using existing calibrated data determines the user's blood characteristics such as the value of the haemoglobin, bilirubin and oxygen saturation of the user's blood from the signal. Lastly, these values of user's health parameters are stored in the cloud for future references.

Another embodiment of the present disclosure is a method of working of a non-invasive portable system includes, passing a light, by a light ring in a device, to the inner side of the ring finger of a user. The method includes reflecting the light at 90 degrees back from the ring finger to the photo diode of the light ring. The method includes producing a spectrum by the photo diode, for example, the photo diode captures more than hundreds of signals continuously with a minimum delay of 500 milliseconds. The method includes determining an average of the signals by the microcontroller of the device and then amplifying the signals through the ADC to obtain a spectrum of signal. The method includes sending the signal to the platform by the device on the mobile device for further processing, say via Bluetooth.

The method includes comparing the signal with the reference spectrum of the database in the platform and then feeding the compared result into the data model. The method includes determining the user's blood characteristics such as the value of the haemoglobin, bilirubin and oxygen saturation from the signal using the data model having probability factors using existing calibrated data. Lastly, the method includes storing these values of user's health parameters in the cloud for future references.

FIG. 1 illustrates an apparatus (100) for determining characteristics of a fluid, in accordance with the present disclosure. In an embodiment, the apparatus (100) includes a visible light producing LED array, an optical system, and a microcontroller. In the apparatus (100), the optimal system (200) includes a grating (106) to receive irradiated light from the object through a collimator (108) and disperse the light into wavelengths. In the apparatus (100), a linear image sensor (102) arranged at a focal plane of the focusing lens to convert the light by the grating and focused by the focusing lens, into electrical signals. In the apparatus (100), the microcontroller connected to the sensor to process the electrical signals and communicate for processing.

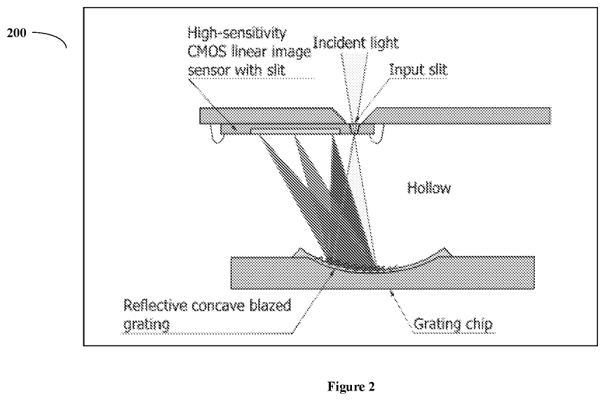

FIG. 2 illustrates working of an optical system (200) in the apparatus, in accordance with the present disclosure. In an embodiment, the visible light producing LED array corresponds to a white LED ring, a combination of 6 LEDs of luminous intensity of 18 mcd placed angularly to produce a concentrated light beam as shown. The LED array is defined by 440-660 nm and a color temperature 7000 K.

In an embodiment, the LED array is configured to pass a visible white light to an inner side of a ring finger of a subject or user. The white light penetrates through a finger-tip by passing epidermis and contacts a concentrated peripheral blood

In an embodiment, as shown in FIG. 2, the grating (106) is used which is a spectral analyzer of 340 to 850 nm, 288 pixels based with 15 nm resolution. The grating (106) is configured to distribute the reflected light from the object to whole spectra from 310 to 850 nm.

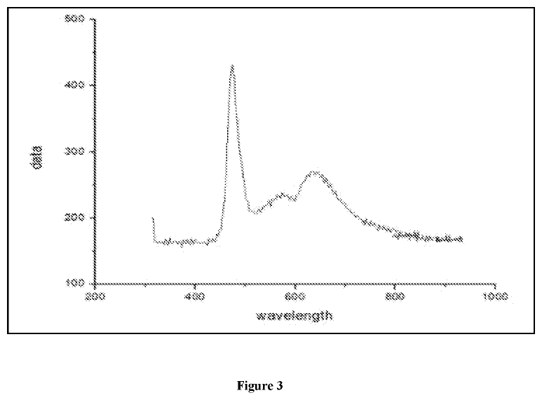

FIG. 3 illustrates a digital diagram of the electrical signals obtained as an output of the apparatus, in accordance with the present disclosure. In an embodiment, the image sensor (102) converts the light which were dispersed into wavelengths by the grating (106) and focused by the focusing lens, into electrical signals. The electrical signals are then converted in digital form in a distributed spectrum as shown in FIG. 3.

In an embodiment, the apparatus (100) includes a remote application which collects the digital signals from microcontroller and processes the signals through signal processing technique.

In an embodiment, the apparatus (100) includes a remote server trained on datasets to collect the processed signal and predict a value for the fluid defined by one or more of hemoglobin, bilirubin, oxygen saturation, createnine and random blood glucose.

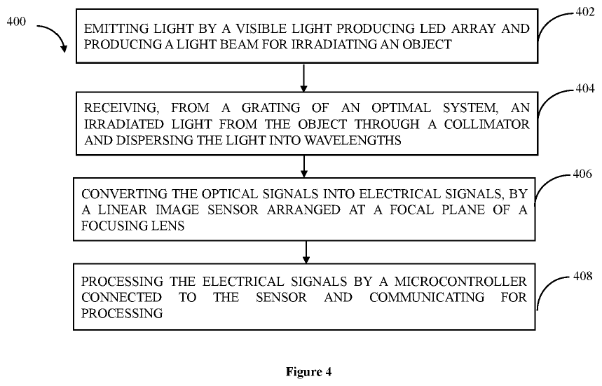

FIG. 4 illustrates a method (400) of determining characteristics of a fluid, in accordance with the present disclosure. At step (402), the method (400) includes emitting light by a visible light producing LED array and producing a light beam for irradiating an object. At step (404), the method (400) includes receiving, from a grating of an optimal system, an irradiated light from the object through a collimator and dispersing the light into wavelengths. At step (406), the method (400) includes converting the optical signals into electrical signals, by a linear image sensor arranged at a focal plane of a focusing lens on the optimal system by the grating and focussing by the focusing lens. At step (408), the method (400) includes processing the electrical signals by a microcontroller connected to the sensor and communicating for processing.

In an embodiment, the method includes passing a visible white light by the LED array to an inner side of a ring finger of a subject and penetrating through a finger-tip by passing epidermis and contacting a concentrated peripheral blood. The LED array is defined by 440-660 nm and a color temperature 7000 K. The LED array corresponds to a white LED ring, a combination of 6 LEDs of luminous intensity of 18 mcd placed angularly to produce a concentrated light beam.

In an embodiment, in the method, the grating used is a spectral analyzer of 340 to 850 nm, 288 pixels based with 15 nm resolution. In the method, the grating configured to distribute the reflected light from the object to whole spectra from 310 to 850 nm.

In an embodiment, the method includes collecting, by a remote application, the digital signals from microcontroller and processing the digital signals through signal processing technique. In an embodiment, the method includes collecting the processed signal by a remote server trained on datasets and predicting a value for the fluid defined by one or more of hemoglobin, bilirubin, oxygen saturation, createnine and random blood glucose.

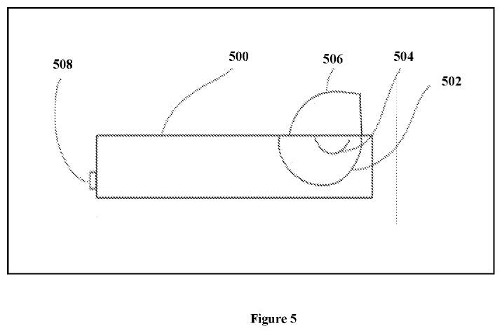

FIG. 5 illustrates an example embodiment of the apparatus, in accordance with the present disclosure. The apparatus is shown in the form of a device (500) which includes a light source (502), a finger bed (504), a cap (506) and a switch (508).

In the device, a user is required to keep ring finger on the finger bed (504) which is covered by the cap (506). On pressing the switch (508), the light sources (502) pass a visible white light to the inner side of the left hand ring finger of the user. The ring finger is the thinnest finger and is selected for medical in vitro diagnosis. Then light penetrates through the finger tip by passing the epidermis and touch the concentrated peripheral blood. The light is then reflected and converted into a electrical signal which sent for processing. The signal is the processed by a remote server trained on datasets and predicting a value for the fluid defined by one or more of hemoglobin, bilirubin, oxygen saturation, createnine and random blood glucose.

The clinical trials of around 12,000 subjects prove that based on the concentration of various biomarkers, the pattern of output signal of the image sensors are varied according to their vitals.

After completed the training datasets having output signals of 12,000 subjects versus actual blood parameters values (i.e., hemoglobin, bilirubin, oxygen saturation, createnine and random blood glucose) showed the biomarker changes and a signal to classify and calculate the actual values of the blood parameters based on the historic training data sets is obtained.

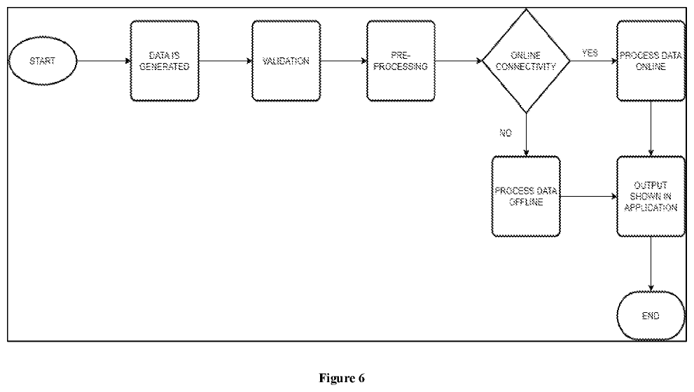

FIG. 6 illustrates the data processing steps for generation to output, in accordance with the present disclosure. All the data is first checked for validity, by checking a few conditions. The training data is pre-processed by removing all the erroneous values, normalized between the maxima and the minima, and noise is removed by using rolling averages. The testing data is also pre-processed similar to the training data. This processing is done to ensure that the data model doesn't output erroneous values of the various parameters and the outputs of the parameters must be within a valid range. This data model is then deployed in the remote application and a cloud server for working with the real-time data. The real-time data also undergoes the same validation and pre-processing steps like the training and testing data to minimize errors. In addition, averaging of multiple data sets is performed to clean the data further. The data is then processed by the cloud data model if internet connectivity is there in the phone, otherwise the data is processed in the android application.

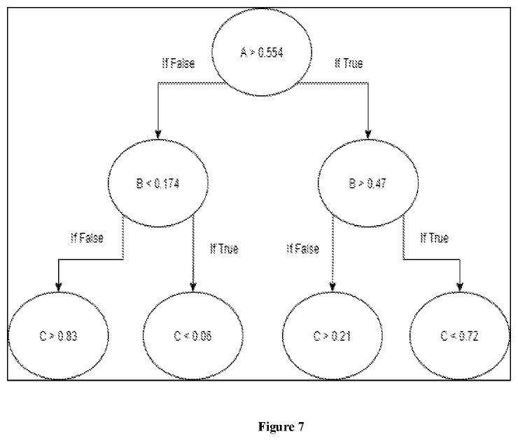

In addition, the data model for this device is based on an auto-generated series of if-then rules, which modifies the values of multiple variables of a calculation as shown in FIG. 7. The output of the calculation gives the various parameters of the device, such as the hemoglobin, bilirubin etc. In an example embodiment, multiple set of if-then rules, all of which focus on the different portions of the same data, are generated by modeling the training data and checking which set of rules gives the most accurate results in the testing data. The data model is then tuned to focus on the critical portion of the data and areas where the accuracy is most important. The data model is further optimized for minimal bias, variance and noise in the output of the calculation.

While specific language has been used to describe the present disclosure, any limitations arising on account thereto, are not intended. As would be apparent to a person in the art, various working modifications may be made to the method in order to implement the inventive concept as taught herein. The drawings and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment.

We claim:

1. An apparatus (100) for determining characteristics of a fluid:
a visible light producing LED array to emit light and produce a light beam for irradiating an object;
an optimal system comprising:
a grating (106) to receive irradiated light from the object through a collimator (108) and disperse the light into wavelengths;
a focusing lens (104);
a linear image sensor (102) arranged at a focal plane of the focusing lens to convert the light by the grating and focused by the focusing lens, into electrical signals;
a microcontroller (102) connected to the sensor to process the electrical signals and communicate the electrical signals

2. The apparatus as claimed in claim 1, wherein the LED array is defined by 440-660 nm and a color temperature 7000 K.

3. The apparatus as claimed in claim 1, wherein the grating (106) is defined by a spectral analyzer of 340 to 850 nm, 288 pixels based with 15 nm resolution and configured to distribute the reflected light from the object to whole spectra from 310 to 850 nm.

4. The apparatus as claimed in claim 1, wherein the LED array corresponds to a white LED ring, a combination of 6 LEDs of luminous intensity of 18 mcd placed angularly to produce a concentrated light beam.

5. The apparatus as claimed in claim 1, further comprising a remote application which collects the digital signals from microcontroller and processes the signals through signal processing technique.

6. The apparatus as claimed in claim 5, further comprising a remote server trained on datasets to collect the processed signal and predict a value for the fluid defined by one or more of hemoglobin, bilirubin, oxygen saturation, createnine and random blood glucose.

7. The apparatus as claimed in claim 1, wherein the LED array is configured to pass a visible white light to an inner side of a ring finger of a subject and penetrate through a finger-tip by passing epidermis and contact a concentrated peripheral blood.

8. A method (400) of determining characteristics of a fluid:
emitting (402) light by a visible light producing LED array and producing a light beam for irradiating an object;
receiving (404), from a grating (106) of an optimal system, an irradiated light from the object through a collimator (108) and dispersing the light into wavelengths;
converting (406) the light into electrical signals, by a linear image sensor (102) arranged at a focal plane of a focusing lens in the optimal system by the grating and focusing by the focusing lens;
processing (408) the electrical signals by a microcontroller (102) connected to the sensor and communicating the electrical signals.

9. The method as claimed in claim 8, wherein the LED array is defined by 440-660 nm and a color temperature 7000 K.

10. The method as claimed in claim 8, wherein the grating (106) is defined by a spectral analyzer of 340 to 850 nm, 288 pixels based with 15 nm resolution and configured to distribute the reflected light from the object to whole spectra from 310 to 850 nm.

11. The method as claimed in claim 8, wherein the LED array corresponds to a white LED ring, a combination of 6 LEDs of luminous intensity of 18 mcd placed angularly to produce a concentrated light beam.

12. The method as claimed in claim 8, further comprising:
collecting, by a remote application, the digital signals from microcontroller and processing the digital signals through signal processing technique.

13. The method as claimed in claim 12, further comprising:
collecting the processed signal by a remote server trained on datasets and predicting a value for the fluid defined by one or more of hemoglobin, bilirubin, oxygen saturation, createnine and random blood glucose.

14. The method as claimed in claim 8, further comprising:
passing a visible white light by the LED array to an inner side of a ring finger of a subject and penetrating through a finger-tip by passing epidermis and contacting a concentrated peripheral blood.
